(19) United States
(12) Patent Application Publication
(10) Pub. No.: US 2005/0118108 A1
(43) Pub. Date: Jun. 2, 2005

(54) Pulmonary delivery of a liquid medicament aerosol

(76) Inventors: Ada S. Cowan, Lewis Center, OH (US); Donna T. Palmer, San Diego, CA (US)

Correspondence Address: Patricia A. Coburn;BattellePharma, Inc., Suite 100, 1801 Watermark Drive, Columbus, OH, 43215-1037, US

(21) Appl. No.: 10/722,763

(22) Filed: Nov. 28, 2003

(51) Int. Cl. A61L 9/04

(52) U.S. Cl. 424/45

(57) ABSTRACT

The invention is directed to liquid compositions containing a drug and liquid carrier vehicles for direct pulmonary delivery of a pharmaceutically active agent to a patient in need of treatment wherein the liquid carrier vehicle consists primarily of a fluorocarbon liquid and a co-solvent such as ethanol and to methods of administering such liquid compositions using an electrohydrodynamic (EHD) aerosolization/spraying means.

### BACKGROUND OF THE INVENTION

1. Field of the Invention

This invention relates to composition and methods for direct pulmonary delivery of a pharmaceutically active agent to the respiratory tract of a patient using an electrohydrodynamic (EHD) spraying/aerosolization means.

2. Description of the Related Art

Delivery of drugs to the lungs of a patient by inhalation of an aerosol is well known in the art. Many different drugs are administered by inhalation; however, the greatest number of inhaled drugs are used to treat condition of the lungs such as asthma, chronic obstructive pulmonary disease (COPD) and emphysema.

Formulations for therapeutic agents delivered by typical hand-held inhalers such as metered dose inhalers and dry powder containers generally contain a halocarbon propellant. Chlorofluorocarbon propellants as well and some fluorocarbon propellants can have adverse environmental effects. For some therapeutic agents, delivery of an aerosolized liquid without a propellant might be preferred.

U.S. Pat. No. 6,261,539, and U.S. Pat. No. 6,004,537 are examples of inhalable formulations containing a drug useful for treating a respiratory disease where the drug is dissolved or suspended in a liquid carrier vehicle and where the formulation also contains a fluorocarbon propellant. The formulated drug and propellant are filled into a container under pressure.

In addition to liquid carrier vehicles, dry powder carrier vehicles are also known as described in U.S. Pat No. 5,875,776. Typically, these dry powders are filled into canisters under pressure and a fluorocarbon or chlorofluorocarbon is used as the propellant.

Pulmonary drug delivery devices that use electrohydrodynamic (EHD) spraying are known. Many are unwieldy and require connection to either an alternative current power supply or a large direct current power supply. A portable, hand-held EHD device is described in U.S. Pat. No. 6,397,838.

Fluorocarbon (FC) liquids such as perfluorooctyl bromide (PFOB) also called perflubron are used in liquid breathing and as blood substitutes. U.S. Pat. No. 5,531,219 describes such applications as well as the use of fluorocarbons to facilitate pulmonary drug delivery.

The use of fluorocarbons as anti-inflammatory agents is described in U.S. Pat. No. 5,470,885. The reference teachers that the fluorocarbon droplets may be delivered using a nebulizer or atomizer.

The prior art describes the use of fluorocarbons as propellants in aerosolizable medicament formulations. Liquid perfluorocarbons are used in liquid breathing, as blood substitutes, as contrast agents and as an anti-inflammatory agent. However, formulations suitable for aerosolization using an EHD aerosolization means containing liquid fluorocarbon as the carrier vehicle for an active drug substance where the active drug substance is dissolved or suspended in the liquid carrier vehicle is not known.

It is an object of the invention to provide compositions for direct pulmonary delivery of a drug where the drug is contained in a liquid carrier vehicle comprised of a fluorocarbon and a co-solvent as well as methods for pulmonary delivery of such compositions using an EHD aerosolization means.

### SUMMARY OF THE INVENTION

The invention is directed to a liquid composition for direct pulmonary delivery of a pharmaceutically active agent to a patient in need of treatment comprising:
a) a pharmaceutically effective amount of said active agent; and
b) a liquid carrier vehicle, wherein said liquid carrier vehicle consists essentially of:
   i) from about 30% v/v to about 99% v/v of a liquid fluorocarbon;
   ii) from about 1% v/v to about 70% v/v of a co-solvent;
   iii) from about 0% w/v to about 10% w/v of a phospholipid; and
   iv) from about 0% w/v to about 10% w/v of a pharmaceutically acceptable excipient; wherein said active agent is dissolved or suspended in said liquid carrier vehicle and wherein said liquid composition has a surface tension of from about 15 dyne/cm to about 40 dyne/cm.

The invention is further directed to a method for delivering a pharmaceutically active agent to the respiratory tract of a patient in need of treatment comprising the steps of:
a) preparing a liquid carrier vehicle consisting essentially of:
   i) from about 30% v/v to about 99% v/v of a liquid fluorocarbon;
   ii) from about 1% v/v to about 70% v/v of a co-solvent;
   iii) from about 0% w/v to about 10% w/v of a phospholipid; and
   iv) from about 0% w/v to about 10% w/v of a pharmaceutically acceptable excipient;
b) dissolving or suspending a pharmaceutically effective amount of said active agent in said liquid carrier vehicle;
c) forming an aerosol of said active agent/liquid carrier vehicle mixture using an EHD spraying/aerosolization means; and
d) administering said aerosol to the pulmonary tract of said patient via inhalation of said aerosol;
wherein said liquid carrier vehicle has a surface tension of from about 15 dyne/cm to about 40 dyne/cm.

The invention is further directed to a liquid carrier vehicle for use with an EHD spraying/aerosolization means consisting essentially of:
i) from about 30% v/v to about 99 v/v of a liquid fluorocarbon;
ii) from about 1% v/v to about 70% v/v of a co-solvent;
iii) from about 0% w/v to about 10% w/v of a phospholipid; and
iv) from about 0% w/v to about 10% w/v of a pharmaceutically acceptable excipient;
wherein said liquid carrier vehicle has a surface tension of from about 15 dyne/cm to about 40 dyne/cm.

### BRIEF DESCRIPTION OF THE DRAWINGS

Aspects of the compositions and liquid carrier vehicles of the invention are illustrated by the accompanying drawings. Four sheets of drawings are provided. Sheet one contains FIG. 1 through FIG. 4. Sheet two contains FIG. 5 through FIG. 8. Sheet three contains FIG. 9 and FIG. 10. Sheet four contains FIG. 11 through FIG. 13.

FIG. 1-FIG. 10 are Malvern plots of Example No's 1, 3-4, 7-11, and 13-14 from Table 2 herein.

FIG. 11-FIG. 13 are Malvern plots of Examples No's 15-17 from Table 3 herein. FIG. 14-FIG. 16 are Malvern plots of Example No's 18-20 from Table 3.

### DETAILED DESCRIPTION

The invention is directed to a liquid composition for direct pulmonary delivery of a pharmaceutically active agent to a patient in need of treatment comprising:
a) a pharmaceutically effective amount of said active agent; and
b) a liquid carrier vehicle, wherein said liquid carrier vehicle consists essentially of:
   i) from about 30% v/v to about 99% v/v of a liquid fluorocarbon;
   ii) from about 1% v/v to about 70% v/v of a co-solvent;
   iii) from about 0% w/v to about 10% w/v of a phospholipid; and
   iv) from about 0% w/v to about 10% w/v of a pharmaceutically acceptable excipient;
wherein said active agent is dissolved or suspended in said liquid carrier vehicle and wherein said liquid composition has a surface tension of from about 15 dyne/cm to about 40 dyne/cm.

Among the preferred embodiments of the compositions of the invention is a composition for direct pulmonary delivery of a pharmaceutically active agent to a patient in need of treatment comprising:
a) a pharmaceutically effective amount of said active agent; and
b) a liquid carrier vehicle, wherein said liquid carrier vehicle consists essentially of:
   i) from about 50% v/v to about 95% v/v of a liquid fluorocarbon;
   ii) from about 2.5% v/v to about 50% v/v of a co-solvent;
   iii) from about 0.1% w/v to about 2.0% w/v of a phospholipid; and
   iv) from about 0.1% w/v to about 5.0% w/v of a pharmaceutically acceptable excipient;
wherein said active agent is dissolved or suspended in said liquid carrier vehicle and wherein said liquid composition has a surface tension of from about 15 dyne/cm to about 40 dyne/cm.

Another aspect of the invention is directed to a method for delivering a pharmaceutically active agent to the respiratory tract of a patient in need of treatment comprising the steps of:
a) preparing a liquid carrier vehicle consisting essentially of:
   i) from about 30% v/v to about 99 v/v of a liquid fluorocarbon;
   ii) from about 1% v/v to about 70% v/v of a co-solvent;
   iii) from about 0% w/v to about 10% w/v of a phospholipid;
   iv) from about 0% w/v to about 10%w/v of a pharmaceutically acceptable excipient;
b) dissolving or suspending a pharmaceutically effective amount of said active agent in said liquid carrier vehicle;
c) producing an aerosol of said solution or suspension using an EHD spraying/aerosolization means; and
d) administering said aerosol to the pulmonary tract of said patient via inhalation of said aerosol;
wherein said liquid carrier vehicle has a surface tension of from about 15 dyne/cm to about 40 dyne/cm.

Yet another embodiment of the invention is to a liquid carrier vehicle for use with an EHD spraying/aerosolization means consisting essentially of:
i) from about 30% v/v to about 99 v/v of a fluorocarbon;
ii) from about 1% v/v to about 70% v/v of a co-solvent;
iii) from about 0% w/v to about 10% w/v of a phospholipid; and
iv) from about 0% w/v to about 10% w/v of a pharmaceutically acceptable excipient.
wherein said liquid carrier vehicle has a surface tension of from about 15 dyne/cm to about 40 dyne/cm.

As used herein, the term “pharmaceutically active agent” refers to biologically active agents that are administered to human or animal patients as the active drug substance for treatment of a disease or condition. Such active drug substances are administered to a patient in a “pharmaceutically effective amount” to treat a disease or condition.

A suitable medicament or drug is one which is suitable for administration by inhalation, the inhalation being used for oral and nasal inhalation therapy. Therapeutic categories of drugs include cardiovascular drugs, antiallergics, analgesics, bronchodilators, antihistamines, antitussives, antifungals, antivirals, anticancer, antibiotics, pain medicaments, antiinflammatories, peptides, proteins and steroids.

Particularly suitable drugs include albuterol (also known as salbutamol), atropine, budesonide, cromolyn, epinephrine, ephedrine, fentanyl, flunisolide, formoterol, ipratropium bromide, isoproterenol, pirbuterol, prednisolone, triamcinolone acetonide, salmeterol, amiloride, fluticasone as well as the pharmaceutically acceptable acid addition salts and esters of the foregoing drugs, their hydrates and their other solvates.

Other suitable medicaments for use in the compositions and methods of the invention include, antineoplastic agents, such cisplatin and carboplatin, methotrexate, taxol, mitomycin, bleomycin, vincristine, vinblastine, dactinomycin, daunorubicin, doxorubicin, mithramycin, tamoxifen, etoposide, alpha- and beta-interferon; anti-fungal agents such as ketoconazole, nystatin, and amphotericin B; beta-lactam antibiotics; hormones such as human growth hormone; steroids, e.g., hydrocortisone and prednisone; vitamins e.g., retinoic acid and derivatives such as 13-cis-retinoic acid; peptides, such as insulin, interferons and interleukins; antivirals such as acyclovir, and azidothymidine (AZT); antibiotics such as chloramphenicol and clindamycin; antiinflammatories; opiates; sedatives; and local anesthetics such as lidocaine hydrochloride.

As would be recognized by one skilled in the art, by “pharmaceutically effective amount” is meant an amount of a pharmaceutically active agent having a therapeutically relevant effect on the disease or condition to be treated. A therapeutically relevant effect relieves to some extent one or more symptoms of the disease or condition in a patient or returns to normal either partially or completely one or more physiological or biochemical parameters associated with or causative of the disease or condition. Specific details of the dosage of a particular active drug may be found in its labeling, i.e., the package insert (see 21 CFR § 201.56 & 201.57) approved by the United States Food and Drug Administration.

When a pharmaceutically active agent is added to the liquid carrier a solution is produced if the active agent is soluble in the liquid carrier and a suspension is produced if the active agent is insoluble. The term “suspension” as used herein is given its ordinary meaning and refers to particles of active agent or aggregates of particles of active agent suspended in the liquid carrier. When the active agent is present as a suspension the particles of active agent will preferably be in the nanometer range; e.g. from about 10 nm to about 2500 nm; preferably from about 50 nm to about 1000 nm and more preferably from about 50 nm to about 500 nm. In order to assure formation of good aerosols and aerosol deposition in the lungs, it is important that the particle size of the drug be less than the size of the aerosol droplets.

The particle size of the aerosol droplets produced when the liquid carrier described herein is sprayed with an EHD device will range from about 1 μm to about 50 μm in diameter with the particular size of the aerosol droplet being selected depending on where in the respiratory tract the drug is to be delivered. Generally, if the drug is to be delivered to the deep lung for systemic activity, the particle size of the resulting aerosol will range from about 1 μm to about 8.0 μm and preferably from about 1 μm to about 5.0 μm. If the drug is to be delivered to the mid-lung, the particle size of the resulting aerosol will range from about 2 μm to about 10 μm and preferably from about 5 μm to about 10 μm will be used. If the pharmaceutically active agent is delivered to the oropharangeal region the particle size of the aerosol will generally range from about 2 μm to about 10 μm with a range of from about 5 μm to about 10 μm being preferred. If the drug is to be delivered to the buccal mulosar or to the nares, the particle size of the resulting aerosol will range from about 10 μm to about 50 μm and preferably from about 20 μm to about 30 μm will be used.

Although any pharmaceutically active agent may be formulated in the liquid carrier vehicles of the invention, pharmaceutically active agents which are particularly suited for formulation are those drugs that are unstable in more conventional EHD liquid carrier vehicles such as water, organic liquids, e.g., ethanol or ethanol/water mixtures.

The liquid carrier vehicles of the invention are useful for preparing aerosols for the delivery of pharmaceutically active agents to the “respiratory tract” of a patient using an EHD spraying/aerosolization device. The term “respiratory tract” as used herein includes the upper airways, including the oropharynx and larynx, followed by the lower airways, which include the trachea followed by bifurcations into the bronchi and bronchioli. The upper and lower airways are called the conductive airways. The terminal bronchioli then divide into respiratory bronchioli, which then lead to the ultimate respiratory zone, the alveoli, or deep lung. Gonda, I. “Aerosols for delivery of therapeutic and diagnostic agents to the respiratory tract,” in Critical Reviews in Therapeutic Drug Carrier Systems, 6: 273-313, (1990).

Usually, the deep lung, or alveoli, is the primary target of inhaled therapeutic aerosols for systemic delivery. However, as used herein, the term “respiratory tract” is additionally meant to include administration of the medicament compositions of the invention to the mucosa of the nasal passages and to the mucosa of the bucca. The preferred target for systemic delivery of an aerosol of an active agent is the deep lung or alveoli.

Dispensing devices are known which produce a finely divided spray of liquid droplets by electrostatic means, more properly referred to as electrohydrodynamic (“EHD”) means. Electrohydrodynamic sprayers have found use in many areas of industry, in medicine for the administration of medicaments by inhalation, in agriculture for crop spraying, and in the automobile industry for paint spraying.

In a typical EHD device, a fluid delivery means delivers fluid to be aerosolized to a nozzle maintained at high electric potential. One type of nozzle used in EHD devices is a capillary tube that is capable of conducting electricity. An electric potential is placed on the capillary tube which charges the fluid contents such that, as the fluid emerges from the tip or end of the capillary tube, a so-called Taylor cone is formed. This cone shape results from a balance of the forces of electric charge on the fluid and the fluid's own surface tension. Desirably, the charge on the fluid overcomes the surface tension and at the tip of the Taylor cone, a thin jet of fluid forms and subsequently and rapidly separates a short distance beyond the tip into an aerosol. Studies have shown that this aerosol (often described as a soft cloud) has a uniform droplet size and a high velocity leaving the tip but that it quickly decelerates to a very low velocity a short distance beyond the tip.

EHD sprayers produce charged droplets at the tip of the nozzle. Depending on the use, these charged droplets can be partially or fully neutralized (with a reference or discharge electrode in the sprayer device) or not. When the EHD device is used to deliver therapeutic aerosols, it is preferred that the aerosol be completely electrically neutralized prior to inhalation by the user to permit the aerosol to reach the pulmonary areas where the particular therapeutic formulation is most effective. However, if nasal deposition of the aerosol is desired, an EHD sprayer without means for discharging or means for partially discharging an aerosol might be preferred since the aerosol would have a residual electric charge as it leaves the sprayer so that the droplets would be attracted to and tightly adhere to the surface of the nares.

Various EHD devices are known in the art, for example, U.S. Pat. No. 6,302,331, U.S. Pat. No. 6,105,877, U.S. Pat. No. 6,457,470, U.S. Pat. No. 6,386,195, U.S. Pat. No. 6,252,129 and U.S. Pat. No. 6,595,208. Although, the various patents disclose different methods for obtaining therapeutic aerosols having an aerosol droplet size of in the range of from 0.1 um to 50 um, very little direction is provided regarding suitable carrier liquids for use in the pulmonary administration of therapeutic agents as solutions or suspensions using EHD spraying/aerosolization devices.

When the co-solvent is miscible with the liquid fluorocarbon and the active drug substance is soluble in the co-solvent the liquid composition of the invention is a solution. When the liquid carrier vehicle of the invention contains a co-solvent which is not miscible with the liquid fluorocarbon a two phase system is formed: a continuous phase consisting of the liquid fluorocarbon and a discontinuous phase made up of the co-solvent or mixtures of co-solvents. The active pharmaceutical agent may be dissolved in the discontinuous phase liquid or suspended in either the continuous phase or discontinuous phase liquid. If the active agent is soluble in the co-solvent a solution will be formed which is dispersed through out the continuous phase liquid. If the active pharmaceutical agent is insoluble in the co-solvent it may be suspended in either the co-solvent or the fluorocarbon.

The term “liquid carrier” as used herein refers to the liquid vehicle in which the active agent to be administered to the patient is dissolved or suspended and which has a surface tension of from about 15 dyne/cm to about 40 dyne/cm. The liquid carrier vehicle consists essentially of the following components:
i) from about 30% v/v to about 99 v/v of a liquid fluorocarbon;
ii) from about 1% v/v to about 70% v/v of a co-solvent;
iii) from about 0% w/v to about 10% w/v of a phospholipid; and
iv) from about 0% to about 10% w/v of a pharmaceutically acceptable excipient.

The term “fluorocarbon” (FC) as used herein refers to hydrofluorocarbons (“HFCs”) as well as perfluorocarbons (“PFCs”). The FCs which are useful herein are liquid at room temperature, have low surface tension, low viscosity and are chemically and thermally inert and are biostatic. The liquid fluorocarbons useful herein are metabolically inert and cannot be broken down in the body. The FCs are also biologically inert and therefore, do not pose toxicological risks from metabolic degradation. FCs are excreted from the body by exhalation.

Fluorocarbons useful in the present invention are a family of compounds containing carbon, fluorine and hydrogen or carbon and fluorine which are volatile, linear, branched chain or cyclic C₁-C₁₀ alkanes or C₂-C₁₀ alkenes having some or all of the hydrogen atoms on the alkane or alkene moiety replaced by fluorine.

HFCs found to be useful herein include 1,1,1,3,3-pentafluorobutane, 1,1,1,3,3-pentachlorobutane, 1,1-dichloro-1,3-difluorobut-2-ene, 1-chloro-1,1,3-trifluorobut-ene, perfluorobutylethane, hexafluoroisobutylene, and hexafluoroisopropanol; especially preferred is perfluorobutylethane. PFCs useful herein include perfluoromethane, perfluoroethane, perfluoropropane, perfluorobutane, perfluorocyclobutane, perfluoropentane, perfluorohexane, perfluorooctane, perfluorooctyl bromide (perflubron), perfluorooctyl iodide, perfluorodecalin and perfluoronapthalene. The PFCs that are preferred for use herein are perfluorooctyl bromide and perfluordecalin.

The liquid carrier vehicles of the invention will contain from about 30% v/v to about 99% v/v of a FC or a mixture of FCs. Preferably the liquid carrier vehicle of the invention will contain from about 50% v/v to about 95% v/v of a FC or a mixture of FCs and more preferably from about 65% v/v to about 95% v/v of a FC or a mixture of FCs.

Depending on the pharmaceutically active agent used in the formulation of the invention, it may be advantageous to include a co-solvent in the liquid carrier vehicles of the invention. The function of the co-solvent is to modify the physical and electrical properties of the fluorocarbon component to bring charges into the liquid so as to be sprayable by EHD spraying/aerosolization means. The co-solvent may also increase compatibility of the active agent with the highly inert FCs.

The co-solvent may be selected from the group consisting of alcohols, ethers, alkyl sulfoxides and combinations thereof. In particularly preferred embodiments the co-solvent will be ethanol. The co-solvent will be present in the liquid carrier vehicle of the invention at from about 1% v/v to about 70% v/v and preferably from about 5% to about 50% v/v, and more preferably about 5% to about 35% v/v.

The compositions and liquid carrier vehicles of the invention may optionally contain a phospholipid surface active agent to help modify the physical properties of the FCs in the formulation. Pharmaceutically acceptable phospholipid surfactants useful in the liquid carrier vehicles are such phospholipids as phosphatidic acid, phosphatidylethanolamine, lecithin, phosphatydylglycerol, diphosphatydylglycerol, dipalmitoylphosphatidylcholine (DPPC), 1-palmitoyl-2-oleoyl-phosphatidylglycerol (POPG), dipalmitoylphosphatidylglycerol (DPPG), as well as mixtures of such phospholipids. Such phospholipids are commercially available, see for example, “Avanti Polar Lipids Products Catalog Edition VI Revised”, from Avanti Polar Lipids, Inc., 700 Industrial Park Drive, Alabaster, Ala. 35007-9105 U.S.A.

The compositions and liquid carrier vehicles of the invention may contain up to about 10% w/v of a phospholipid surface active agent; preferably from about 0.1% to about 2% w/v, and more preferably about 0.1% to about 1% w/v phospholipid surfactant will be used.

Other optionally present components in the compositions and liquid carrier vehicles of the invention are “pharmaceutically acceptable excipients”. Pharmaceutically acceptable excipients are those compounds recognized by the FDA as being safe for use in humans. Additives such as polyols e.g., propylene glycol, glycerol, polyvinyl alcohol (PVA)) and polyethylene glycol (PEG) having an average molecular weight between about 200 and 4000, antioxidants, e.g., Vitamin E, Vitamin E TPGS (alpha tocopferol polyethylene glycol 1000 succinate), ascorbic acid, anti-microbials, e.g, parabens, pH adjusting agents, e.g., sodium hydroxide and hydrochloric acid, viscosity adjusting agents, e.g., polyvinyl pyrrolidone and ionic materials to add charge to the liquid carrier formulation are contemplated for use herein.

The compositions liquid carrier formulations of the invention may include minor amounts, that is, up to 10% w/v, preferably from about 0.05% to from about 5% and more preferably from about 0.1 to about 2.5% w/v of a “pharmaceutically acceptable excipient”.

As used herein, the term “pharmaceutically acceptable excipient” refers not only to a single excipient but also to mixtures of two or more excipients; e.g., a composition or liquid carrier vehicle of the invention might contain an antioxidant, a viscosity adjusting agent and an ionic material.

Since the liquid compositions and liquid carrier vehicles of the invention consist primarily of a fluorocarbon liquid as described herein, they are largely inert, insulative and nonconducting. In order to get the liquid carrier vehicle to spray and aerosolize using an EHD device one must be able to add a charge to the carrier liquid sufficient to overcome the surface tension of the carrier liquid as it exits the spray nozzle of the EHD device at a practical voltage, i.e., less than 10 KV. One way to get the carrier liquid to carry a charge is to add charges to the liquid carrier vehicle by addition of a bulky salt such as benzalkonium chloride or a salt such as sodium chloride or potassium chloride. For example, the sodium salt of POPG may be paired with benzalkonium chloride to give charges to the fluorocarbon liquid in the carrier vehicle. Salts such as sodium chloride can dissolve in the co-solvent and add charge to the carrier vehicle. Finally, the active agent may exist as a salt and will be able to add charge to the carrier vehicle.

While the selection of any particular pharmaceutically acceptable excipient is within the skill of the art, the decision regarding whether to add an excipient and if so which one, will be made taking into account the purpose of the excipient in a specific liquid carrier vehicle. In order to be pharmaceutically acceptable any formulation excipient used in the carrier liquids of the invention should be recognized by the FDA as safe for use in humans. Additionally, an excipient should have no negative or minimal negative effect on the sprayability of formulations of a drug in a liquid carrier using an EHD spraying means.

The particle size distribution of a medicament aerosol that is administered to the respiratory tract via inhalation is very important. If delivery to the deep lung is the target, the particle size of the droplets should average about 0.5-5 μm. This average is usually referred to as “mass median diameter” (MMD). In addition to having a MMD of from 0.5-5 μm it is also important that the corresponding geometric standard deviation (GSD) be low to produce a monodisperse aerosol. If the MMD is in the right particle range but the GSD is above about 2, the aerosol will be polydisperse and will contain many aerosol particles that are smaller than 0.5 μm and many that are larger than 5 μm. The result is that the smaller particles may not deposit in the deep lung but may be expired by the patient and the larger particles will be deposited in the upper airways rather than the deep lung. It is thus highly desirable that the aerosol be monodisperse.

The term “(%) RF” used in Table 2 and Table 3 refers to the percent respirable fraction of the aerosol droplets that is less than 5.79 μm. In order to get most of the inhaled dose of drug to the lungs it is important that as much of the dose is inhaled by the patient and delivered to the target site as possible. If the target is the deep lung then most of the dose should have a particle size under about 5.0 μm.

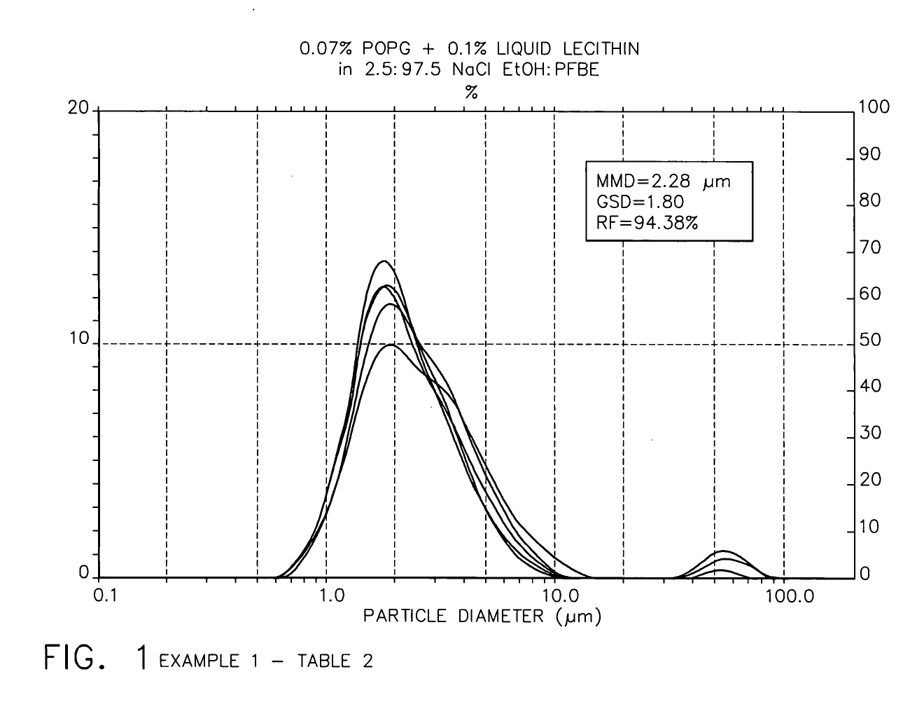

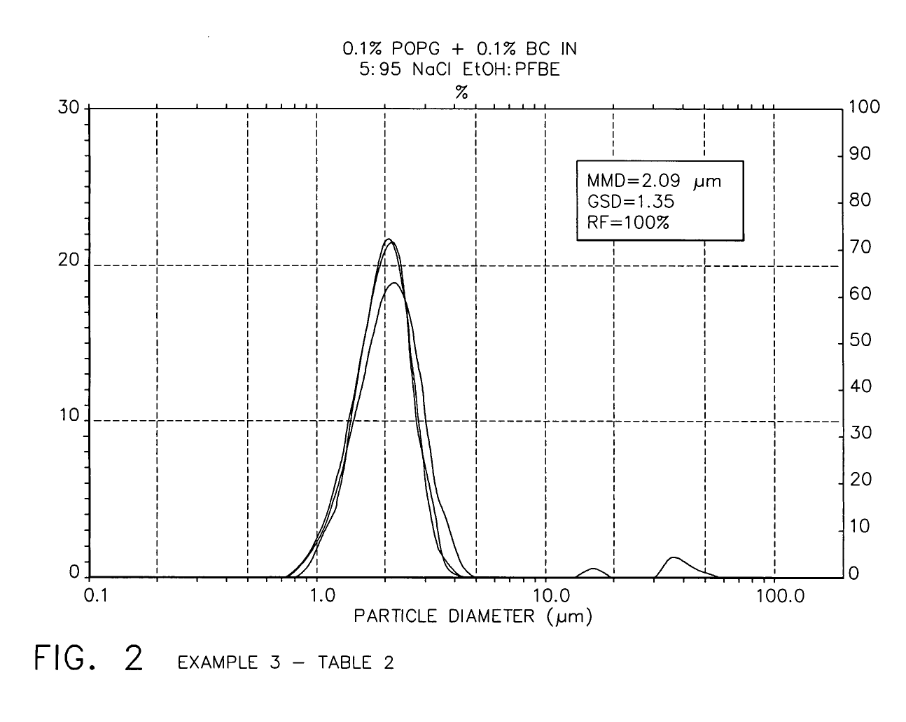

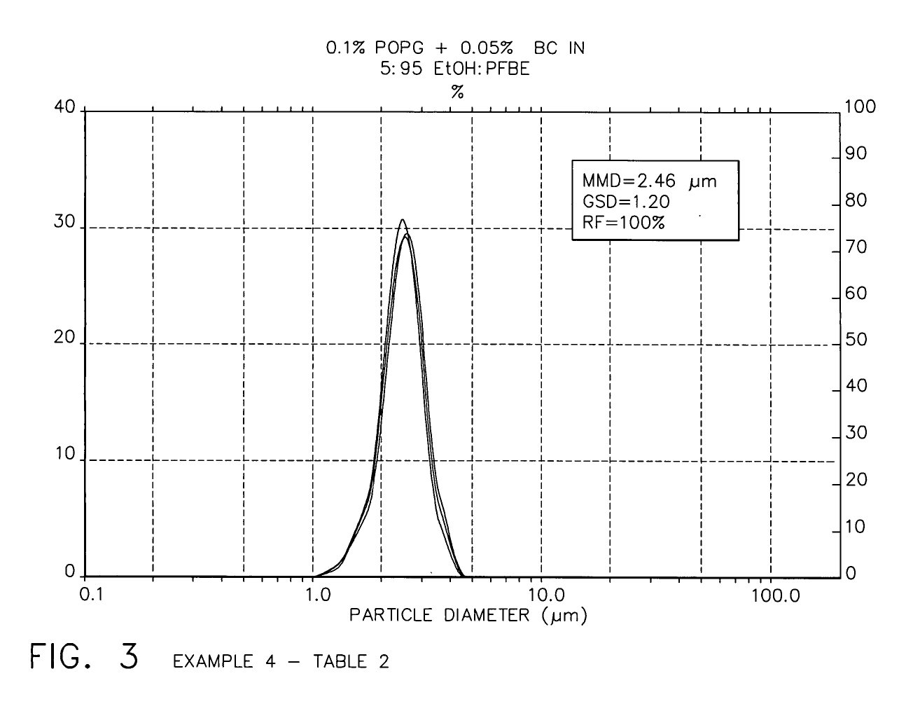

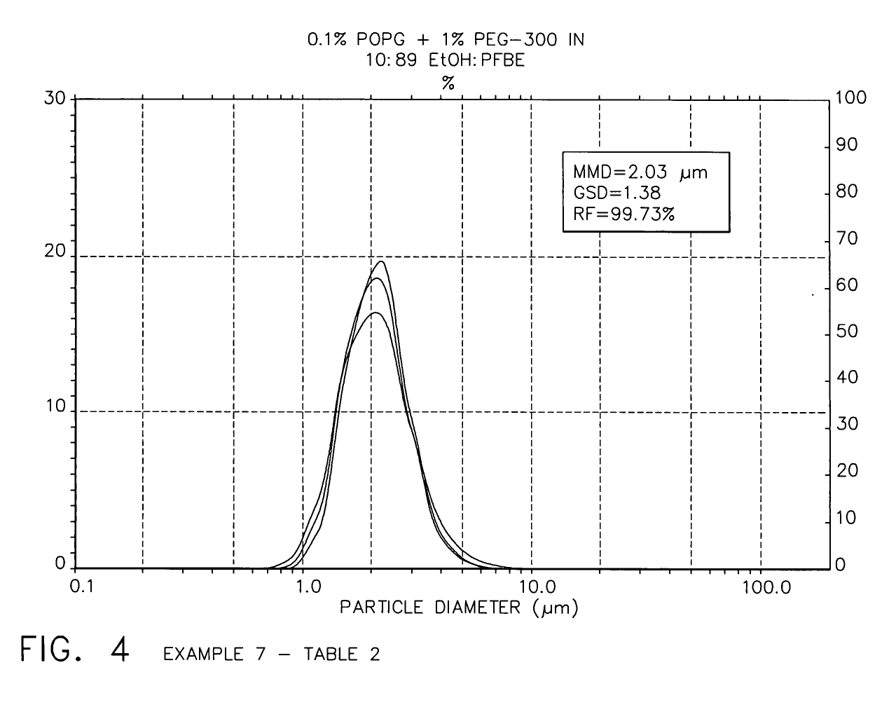

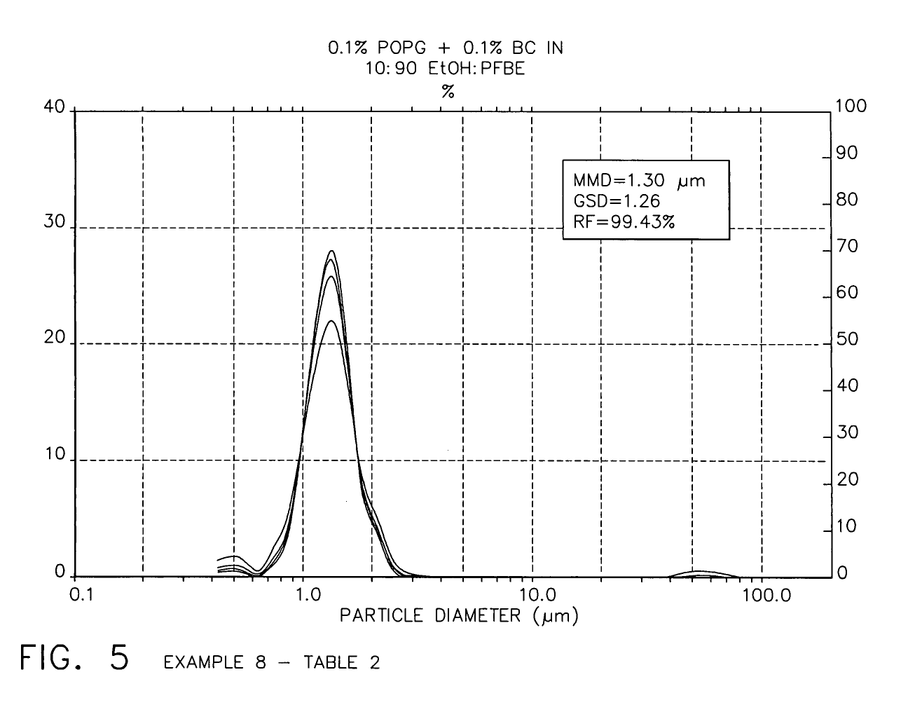

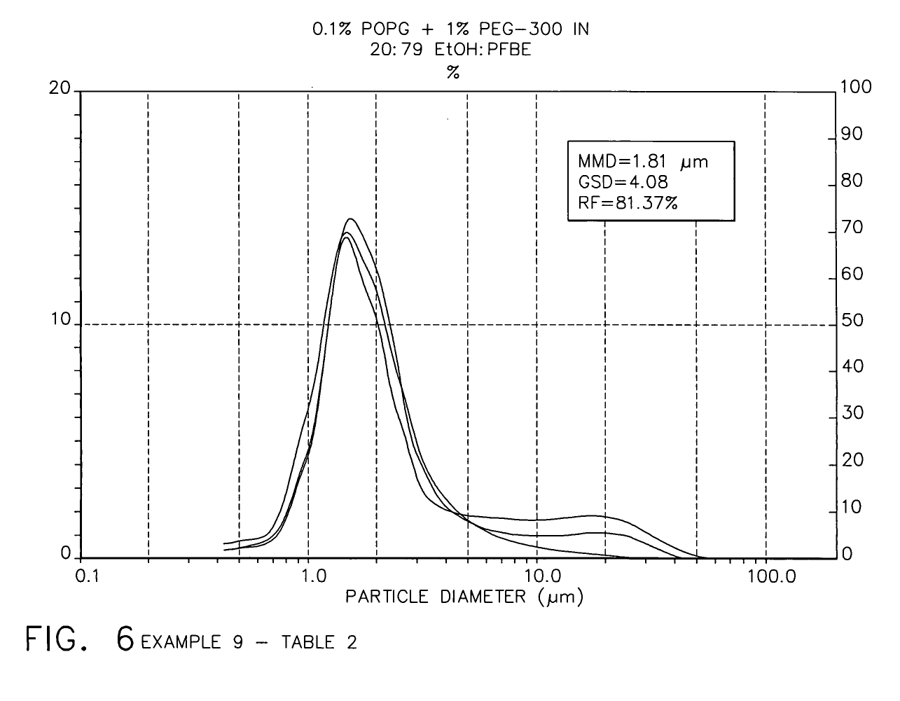

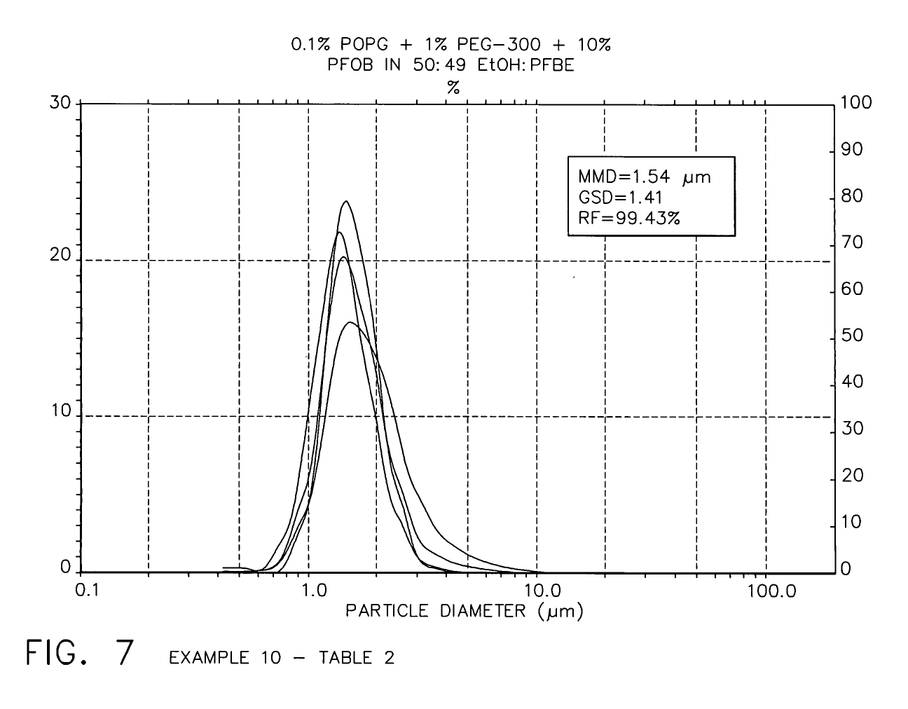

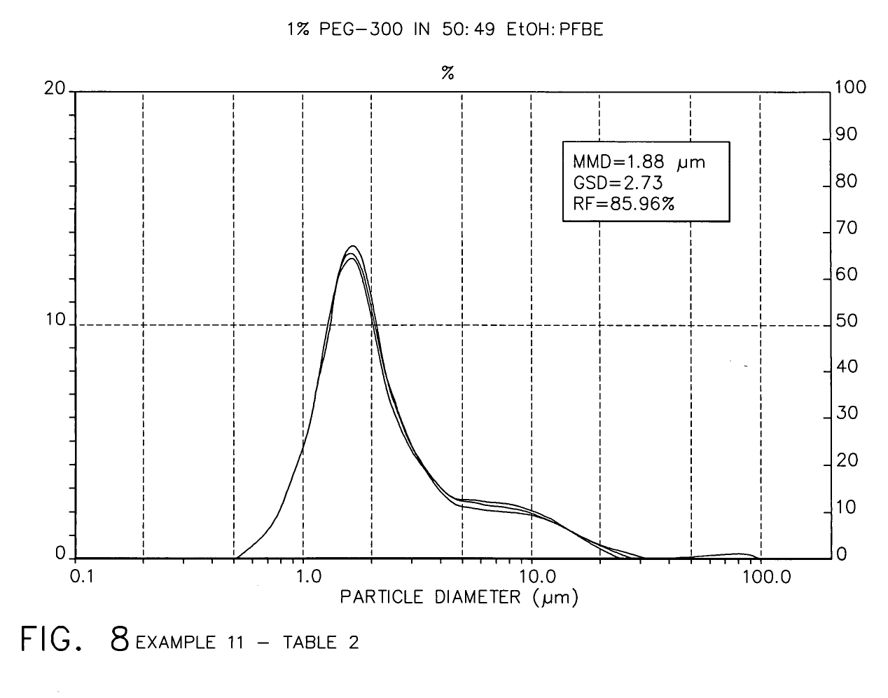

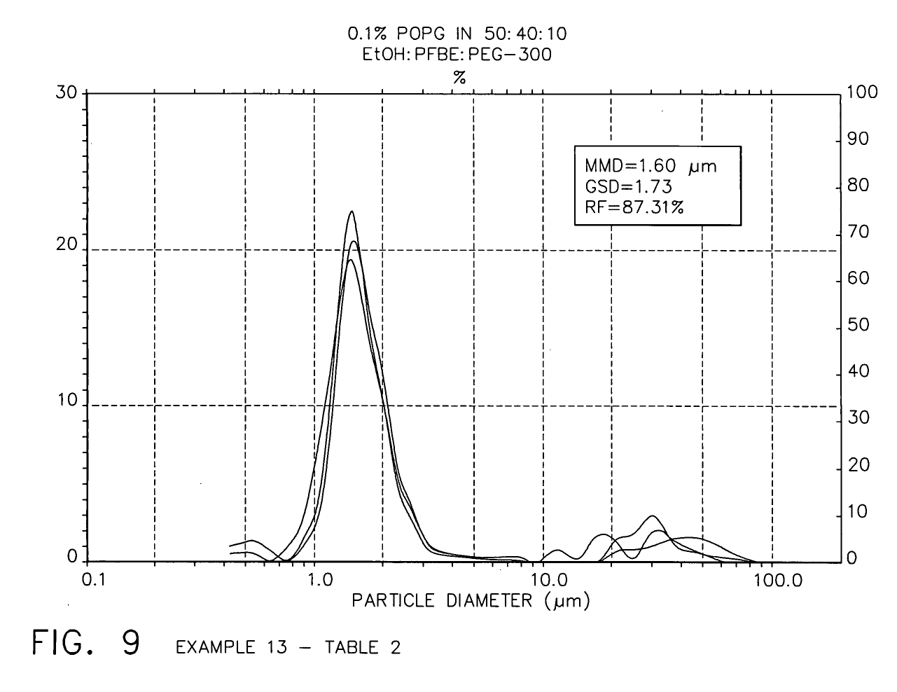

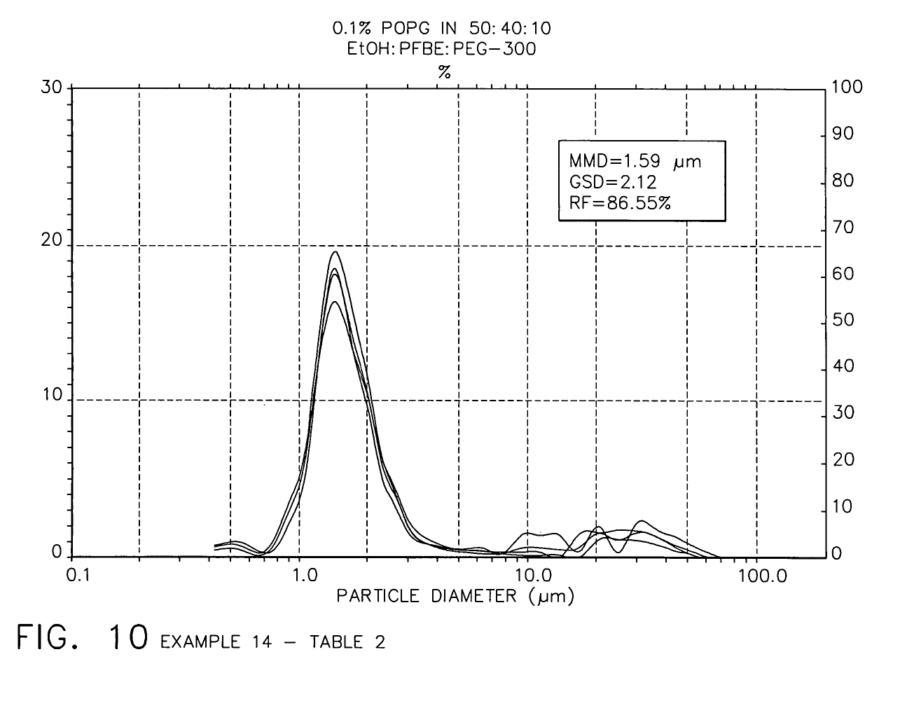

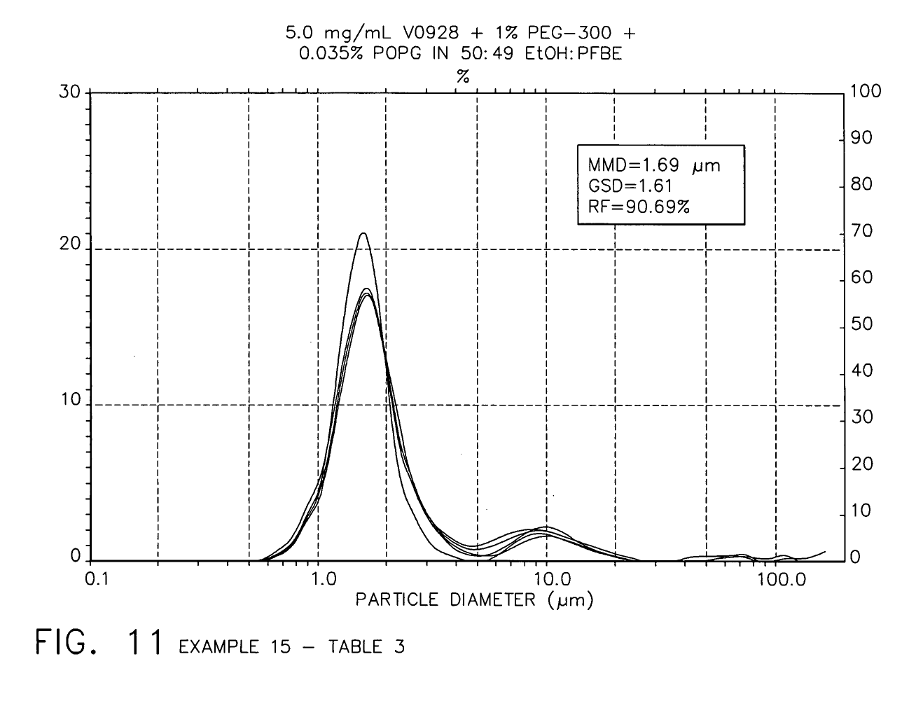

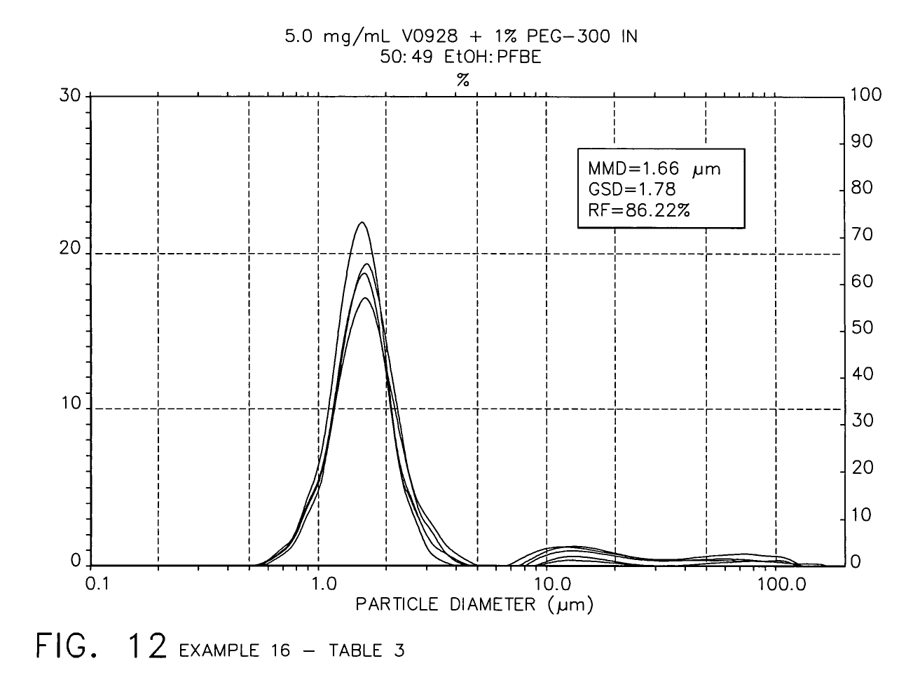

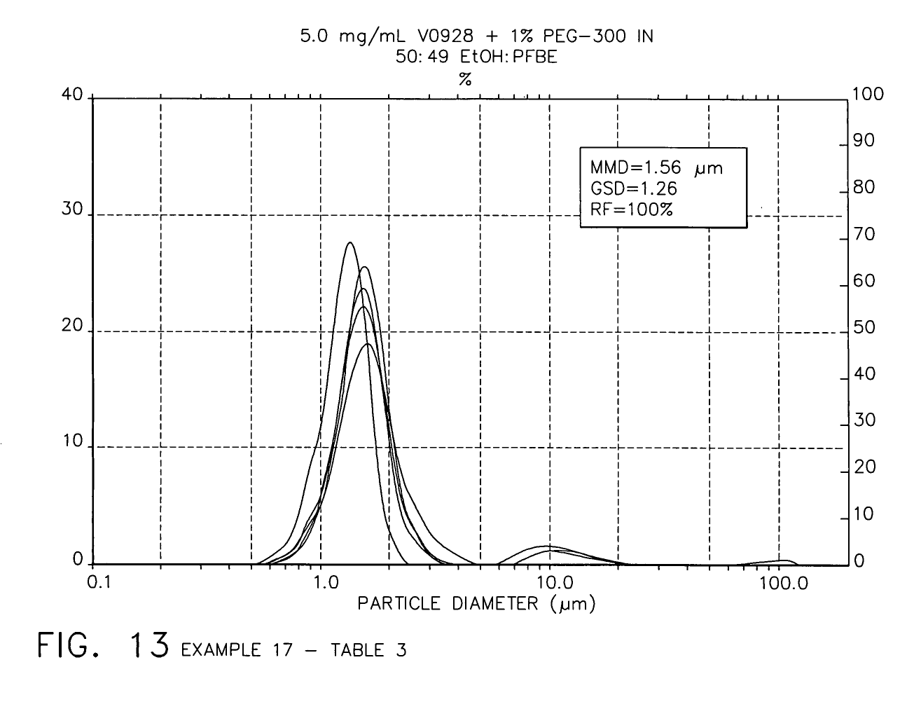

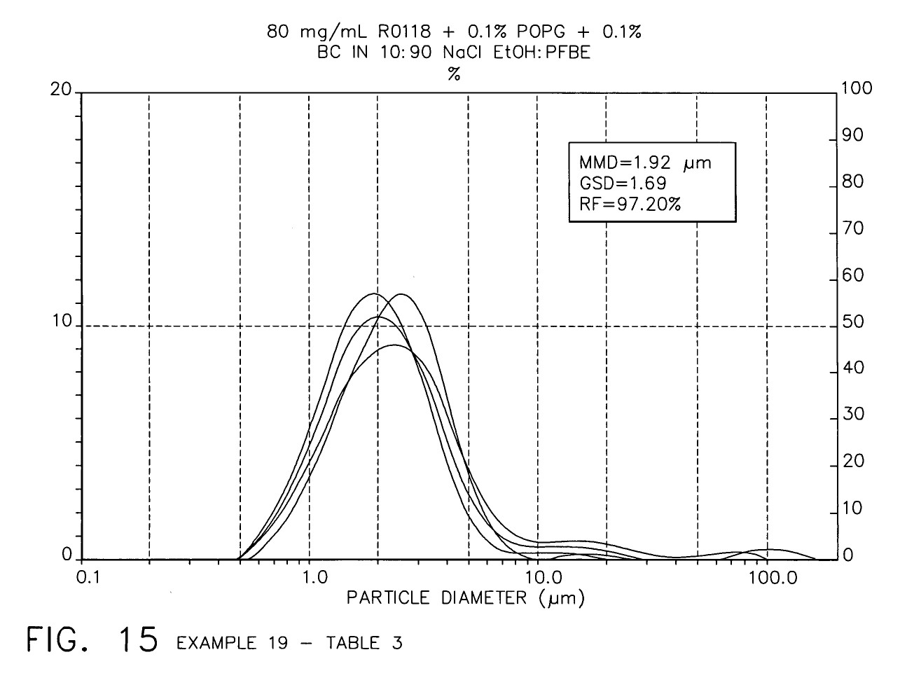

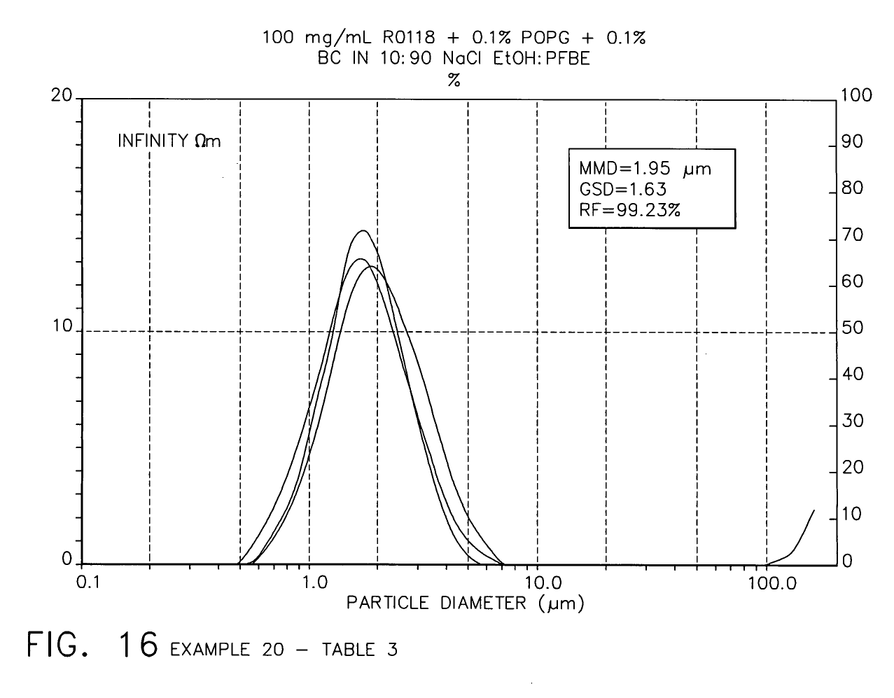

The following examples illustrate the method and various compositions and carrier vehicles described herein. A Glossary of abbreviations is provided for ease of understanding the examples and data presented in the tables. The Malvern Plots shown in FIGS. 1-13 were made using a Malvern MasterSizer X particle size analyzer available from Malvern Instruments Inc., 10 Southville Road, Southborough, Mass. 01772 USA, www.malverninstruments.com.

TABLE 1
Glossary of Abbreviations Used in Examples and Tables

| | | Type of |
|---|---|---|
| Abbreviation | Complete Name | Component |
| EtOH | ethanol | co-solvent |
| POPG | 1-palmitoyl-2-oleoyl- | phospholipid |
| | phosphatidylglycerol | |
| PFOB | perfluorooctylbromide | fluorocarbon |
| PFBE | perfluorobutylethane | fluorocarbon |
| PEG | Polyethylene glycol | excipient |
| BC | Benzalkonium chloride | salt for ionic adjustment |
| PEG-Phos-2K | mPEG-distearoyl- | phospholipid |
| | phosphatidylethanolamine | |

Example 1 describes the preparation of a liquid carrier vehicle of the invention. Table 2 describes additional examples as well as the flow rate, MMD, GSD and % RF of the examples when they were aerosolized using a polymeric circular nozzle array of the type disclosed in U.S. Ser. No. 10/375,957, filed Feb. 28, 2003, entitled “Improved Nozzle for Handheld Pulmonary Aerosol Delivery Device”. In Table 2 and Table 3, the term “KVn” indicates the voltage applied to the nozzle of the EHD device to place a charge on the aerosol droplets and “KVd” indicates the voltage used to discharge the charge on the aerosol droplets. Unless otherwise indicated, the FC used in the Examples listed in Tables 2-3 was PFBE.

### EXAMPLE 1

### Preparation of Liquid Carrier Vehicle

Ten (10) mg of POPG was weighed into a 20 mL ethanol rinsed Type I Teflon-lined screw-cap vial. To the vial was then added 5.0 mL of ethanol. The POPG was dissolved by either or both gentle warming (<40° C.) and brief sonication followed by vortexing; thereafter, 1 mL of PEG-300 was added to the vial with mixing. Finally, 4.0 mL of PFBE was added and the final mixture was briefly vortexed to give a solution. The liquid carrier vehicle prepared in this Example 1 is listed in Table 2 as Sample No. 13. In a similar fashion as described in Example 1 other carrier vehicles were prepared as illustrated by Table 2 below

TABLE 2
Examples of Liquid Carrier Vehicles of the Invention

| | | | | | | Aerosol Characteristics | | |
|---|---|---|---|---|---|---|---|---|
| Example | HFA | | | Flow Rate | Voltages | MMD | | |
| No. | (%) | EtOH | Other Excipients | (μL/s) | (kVn/kVd) | (μm) | GSD | RF_{<5.79} (%) |
| 1 | 97.5 | 2.5% EtOH | 0.07% POPG + 0.1% Liquid Lecithin + | 7 | 7.5/3.5 | 2.28 | 1.80 | 94.38 |
| | | | trace of NaCl | | | | | |
| 2 | 95 | 5% EtOH | 0.07% POPG + 0.2% PEG-300 + 0.1% | 7 | 6.7/2.5 | 2.28 | 1.36 | 98.55 |
| | | | PEG-Phos-2K + trace of NaCl | | | | | |
| 3 | 95 | 5% EtOH | 0.1% POPG + 0.1% BC + trace of NaCl | 8 | 7.1/3.0 | 2.09 | 1.35 | 100 |
| 4 | 95 | 5% EtOH | 0.1% POPG + 0.05% BC | 8 | 7.0/3.4 | 2.46 | 1.20 | 100 |
| 5 | 95 | 5% EtOH | 0.5% POPG + 0.05% BC | 8 | 7.5/2.5 | 2.77 | 1.30 | 99.65 |
| 6 | 90 | 10% EtOH | 0.15% POPG + 0.15% BC + trace of NaCl | 7 | 7.5/2.5 | 1.24 | 1.31 | 100 |
| 7 | 89 | 10% EtOH | 0.1% POPG + 1% PEG-300 | 7 | 6.7/3.4 | 2.03 | 1.38 | 99.73 |
| 8 | 90 | 10% EtOH | 0.1% POPG + 0.1% BC | 7 | 6.7/3.4 | 1.30 | 1.26 | 99.43 |
| 9 | 79 | 20% EtOH | 0.1% POPG + 1% PEG-300 | 7 | 7.2/5.5 | 1.81 | 4.08 | 81.37 |
| 10 | 39 PFBE | 50% EtOH | 0.1% POPG + 1% PEG-300 | 7 | 6.7/3.4 | 1.54 | 1.41 | 99.43 |
| | 10 PFOB | | | | | | | |
| 11 | 49 | 50% EtOH | 1% PEG-300 | 7 | 6.7/4.7 | 1.88 | 2.73 | 85.96 |
| 12 | 49 | 50% EtOH | 1% PEG-300 | 7 | 6.3/3.3 | 1.88 | 3.38 | 82.42 |
| 13 | 40 | 50% EtOH | 0.1% POPG + 10% PEG-300 | 7 | 6.7/3.4 | 1.60 | 1.73 | 87.31 |
| 14 | 40 | 50% EtOH | 0.1% POPG + 10% PEG-300 | 7 | 6.7/3.4 | 1.59 | 2.12 | 86.55 |

FIG. 1 through FIG. 10 are Malvern plots of Samples No. 1, 3-4, 7-11 and 13-14 from Table 2, illustrating the nearly monodisperse nature of the aerosols produced using the liquid carrier vehicles of the invention. The data in Table 2 demonstrates that the aerosols may be produced using a relatively high flow rate, using practical voltages and with a high respirable fraction.

### EXAMPLE 2

### Preparation of Drug Formulation

To an ethanol rinsed 20 mL Type I Teflon-lined screw-cap vial was added 0.1 mL of PEG-300; thereafter, 5.0 mL of ethanol was added. To the resulting mixture was added 4.9 mL of PFBE. Finally, 50 mg (0.5% w/v) of a proprietary small molecule drug was added to the solution. Table 3 illustrates various liquid compositions of the invention wherein the active drug substance is a proprietary small molecule of third parties and whose structure was not disclosed to the inventors of this invention. The composition of the invention prepared in Example 2 is listed as Example 16 and 17 in Table 3.

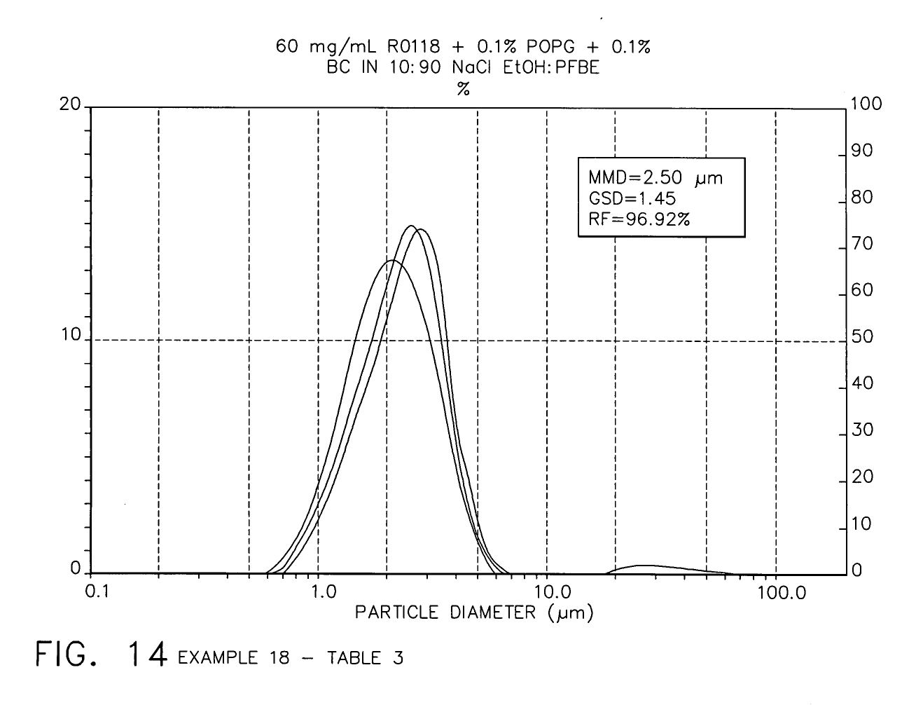

The proprietary compounds have the code designation V0928 and R0118. The composition prepared according to Example 2 is listed in Table 3 as Sample No. 16 and 17. FIGS. 11-13 are Malvern plots of Samples 15-20 from Table 3.The data illustrates that the liquid compositions of the invention are nearly monodisperse in nature and that addition of the active drug substance did not affect sprayability of the liquid.

TABLE 3
Compositions of the Invention

| | | | | | Flow | | Aerosol Characteristics | | |
|---|---|---|---|---|---|---|---|---|---|
| | HFA | | | Drug | Rate | Voltages | MMD | | |
| Ex. No. | (%) | EtOH | Other Excipients | (mg/mL) | (μL/s) | (kVn/kVd) | (μm) | GSD | RF_{<5.79} (%) |
| 15 | 49 | 50% EtOH | 1% PEG-300 + 0.035% | 5 mg/mL | 7 | 6.7/3.4 | 1.69 | 1.61 | 90.69 |
| | | | POPG | V0928 | | | | | |
| 16 | 49 | 50% EtOH | 1% PEG-300 | 5 mg/mL | 7 | 6.5/3.2 | 1.66 | 1.78 | 86.22 |
| | | | | V0928 | | | | | |
| 17 | 49 | 50% EtOH | 1% PEG-300 | 5 mg/mL | 7 | 6.7/3.4 | 1.56 | 1.26 | 100 |
| | | | | V0928 | | | | | |
| 18 | 90 | 10% EtOH | 0.1% POPG + 0.1% BC + trace | 60 mg/mL | 7 | 6.7/3.4 | 2.50 | 1.45 | 96.92 |
| | | | of NaCl | R0118 | | | | | |
| 19 | 90 | 10% EtOH | 0.1% POPG + 0.1% BC + trace | 80 mg/mL | 10 | 6.7/3.4 | 1.92 | 1.69 | 97.20 |
| | | | of NaCl | R0118 | | | | | |
| 20 | 90 | 10% EtOH | 0.1% POPG + 0.1% BC + trace | 100 mg/mL | 10 | 7.2/3.6 | 1.95 | 1.63 | 99.23 |
| | | | of NaCl | R0118 | | | | | |

### EXAMPLE 3

### Fluticasone Propionate Formulation

Into a 20 mL ethanol rinsed Type I Teflon-lined screw-cap vial is added 10 mg of POPG. To the vial is then added 4.5 mL of ethanol. POPG is dissolved by gentle warming (<40° C.) and brief sonication followed by vortexing; thereafter, 1 mL of PEG-300 is added to the vial with mixing. To the vial is added 4.5 mL of PFBE and the mixture is briefly vortexed to give a solution. To the solution is added 50 mg of fluticasone propionate to give a 5 mg/mL concentration of the drug. The resulting suspension should be shaken gently before aerosolization using the aerosolization means described in Example 1.

### EXAMPLE 4

### Budesonide Formulation

To an ethanol rinsed 20 mL Type I Teflon-lined screw-cap vial is added 5 mg of POPG; thereafter, 5 mg BC is weighed into the vial and 0.5 mL of ethanol is added. The POPG and BC is dissolved into the EtOH by gentle warming (<40° C.) and brief sonication followed by vortexing. To the resulting mixture is added 4.5 mL of PFBE. Immediately after PFBE addition, the solution is heated to >50° C. in a hot water bath (95° C.). The solution is then vortexed until the POPG and BC are into solution. Finally, 100 mg of micronized budesonide (particles preferably less than 2 microns) is added to the solution to produce a 20 mg/mL (2% w/v) concentration of budesonide. The resulting suspension should be shaken gently before aerosolization using the aerosolization means described in Example 1.

### EXAMPLE 5

### Albuterol Sulfate Formulation

Into a 20 mL ethanol rinsed Type I Teflon-lined screw-cap vial is added 10 mg of POPG. To the vial is then added 5.0 mL of ethanol. POPG is dissolved by gentle warming (<40° C.) and brief sonication followed by vortexing; thereafter, 1 mL of PEG-300 is added to the vial with mixing. To the solution is added 240 mg of micronized albuterol sulfate (particles preferably less than 2 microns) and the mixture is vortexed. To the suspension is added 5.0 mL of PFBE and 1.0 mL of PFOB and the mixture is vortexed giving a final concentration of albuterol sulfate of 83 μg/mL (2% w/v). The resulting suspension should be shaken gently before aerosolization using the aerosolization means described in Example 1.

The data presented in Tables 2 and 3 illustrates that using the carrier vehicles of the invention in he method of the invention one is able to use relatively high flow rates, i.e., μl/sec as opposed to μl/min of the prior art and low voltages to produce nearly monodisperse aerosols having an MMD of 3 μm or less.

As illustrated by the data presented in Tables 2 and 3, in the practice of the method claimed herein using a handheld EHD inhaler, the best results were achieved when the voltage applied to the EHD nozzle was greater than the voltage applied to the discharge electrode. The particular ratio of KVn to KVd will vary depending on the formulation being sprayed and the particular EHD device being used to create the aerosol.

To the extent necessary to understand or complete the disclosure of the present invention, all publications, patents, and patent applications mentioned herein are expressly incorporated by reference therein to the same extent as though each were individually so incorporated.

The foregoing description is given for clearness of understanding only, and no unnecessary limitations should be understood therefrom, as modifications within the scope of the invention may be apparent to those having ordinary skill in the art. The present invention has been described with reference to particular preferred embodiments; however, the scope of the invention is defined by the following claims and should be construed to include reasonable equivalents.

1. A liquid composition for direct pulmonary delivery of a pharmaceutically active agent to a patient in need of treatment comprising:
a) a pharmaceutically effective amount of said active agent; and
b) a liquid carrier vehicle, wherein said liquid carrier vehicle consists essentially of:
i) from about 30% v/v to about 99% v/v of a liquid fluorocarbon;
ii) from about 1% v/v to about 70% v/v of a co-solvent;
iii) from about 0% wv to about 10% w/v of a phospholipid; and
iv) from about 0% w/v to about 10% w/v of a pharmaceutically acceptable excipient;
wherein said active agent is dissolved or suspended in said liquid carrier vehicle and wherein said liquid composition has a surface tension of from about 15 dyne/cm to about 40 dyne/cm.

2. The composition according to claim 1 wherein said fluorocarbon is present in said carrier vehicle at from about 50% v/v to about 95% v/v.

3. The composition according to claim 2 wherein said fluorocarbon is present in said carrier vehicle at from about 65% v/v to about 95% v/v.

4. The composition according to claim 1 wherein said fluorocarbon is selected from the group consisting of 1,1,1,3,3-pentafluorobutane, 1,1,1,3,3-pentachlorobutane, 1,1-dichloro-1,3-difluorobut-2-ene, 1-chloro-1,1,3-trifluorobut-2-ene, perfluorobutylethane, hexafluoroisobutylene, hexafluoroisopropano, perfluoromethane, perfluoroethane, perfluoropropane, perfluorobutane, perfluorocyclobutane, perfluoropentane, perfluorohexane, perfluorooctane, perfluorooctyl bromide, perfluorooctyl iodide, perfluorodecalin and perfluoronapthalene.

5. The composition according to claim 4 wherein said fluorocarbon is selected from the group consisting of perfluorooctyl bromide and perfluorodecalin.

6. The composition according to claim 5 wherein said fluorocarbon is present in the carrier vehicle at from about 50% v/v to about 95% v/v.

7. The composition according to claim 6 wherein said fluorocarbon is present in said carrier vehicle at from about 65% v/v to about 95% v/v.

8. The composition according to claim 1 wherein said co-solvent is selected from the group consisting of alcohols, ethers and alkyl sulfoxides.

9. The composition according to claim 8 wherein said co-solvent is ethanol.

10. The composition according to claim 9 wherein said ethanol is present in said carrier vehicle at from about 5% v/v to about 50% v/v.

11. The composition according to claim 10 wherein said ethanol is present in the carrier vehicle at from about 5.0% v/v to about 35% v/v.

12. The composition according to claim 3 wherein said ethanol is present in the carrier vehicle at from about 5% v/v to about 35% v/v.

13. The composition according to claim 1 wherein said phospholipid is selected from the group consisting of phosphatidic acid, phosphatidylethanolamine, lecithin, phosphatydylglycerol, diphosphatydylglycerol, dipalmitoylphosphatidylcholine, 1-palmitoyl-2-oleoyl-phosphatidylglycerol, dipalmitoylphosphatidylglycerol, as well as mixtures of such phospholipids

14. The composition according to claim 13 wherein said phospholipid is selected from the group consisting of lecithin, dipalmitoylphosphatidylcholine and 1-palmitoyl-2-oleoyl-phosphatidylglycerol.

15. The composition according to claim 13 wherein said phospholipid is present in the carrier vehicle at from about 0.1% w/v to about 2.0% w/v.

16. The composition according to claim 15 wherein said phospholipid is present in the carrier vehicle at from about 0. 1% w/v to about 1.0 w/v.

17. The composition according to claim 1 wherein said pharmaceutically acceptable excipient is selected from the group comprising polyols, antioxidants, anti-microbials, pH adjusting agents, viscosity adjusting agents and salts.

18. The composition according to claim 17 wherein said pharmaceutically acceptable excipient is selected from the group consisting essentially of propylene glycol, glycerol, polyvinyl alcohol, polyethylene glycol having an average molecular weight between about 200 and 4000, Vitamin E, Vitamin E TPGS, ascorbic acid, methyl paraben, ethyl paraben, sodium hydroxide, hydrochloric acid, polyvinyl alcohol, polyvinyl pyrrolidone, benzalkonium chloride, sodium chloride and potassium chloride.

19. The composition according to claim 17 wherein said pharmaceutically acceptable excipient is present in said carrier vehicle at from 0.05% w/v% to about 5.0% w/v.

20. The composition according to claim 19 wherein said pharmaceutically acceptable excipient is present in said carrier vehicle at from about 0.1% w/v% to about 2.5% w/v.

21. A composition according to claim 1 comprising:
a) a pharmaceutically effective amount of said active agent; and
b) a liquid carrier vehicle, wherein said liquid carrier vehicle consists essentially of:
i) from about 50% v/v to about 95% v/v of a liquid fluorocarbon;
ii) from about 5% v/v to about 50% v/v of a co-solvent;
iii) from about 0.1% w/v to about 2.0% w/v of a phospholipid; and
iv) from about 0.05% w/v to about 5.0% w/v of a pharmaceutically acceptable excipient.

22. A composition according to claim 1 comprising:
a) a pharmaceutically effective amount of said active agent; and
b) a liquid carrier vehicle, wherein said liquid carrier vehicle consists essentially of:
i) from about 65% v/v to about 95% v/v of a liquid fluorocarbon;
ii) from about 5.0% v/v to about 35% v/v of a co-solvent;
iii) from about 0.1% w/v to about 1.0% w/v of a phospholipid; and
iv) from about 0.1% w/v to about 2.5% w/v of a pharmaceutically acceptable excipient.

23. A liquid carrier vehicle for use with an EHD spraying/aerosolization means consisting essentially of:
i) from about 30% v/v to about 99 v/v of a liquid fluorocarbon;
ii) from about 1% v/v to about 70% v/v of a co-solvent;
iii) from about 0% w/v to about 10% w/v of a phospholipid; and
iv) from about 0% w/v to about 10% w/v of a pharmaceutically acceptable excipient.
wherein said liquid carrier vehicle has surface tension of from about 15 dyne/cm to about 40 dyne/cm.

24. The liquid carrier vehicle according to claim 23 wherein said fluorocarbon is present in the carrier vehicle at from about 50% v/v to about 95% v/v.

25. The liquid carrier vehicle according to claim 24 wherein said fluorocarbon is present in said carrier vehicle at from about 65% v/v to about 95% v/v.

26. The liquid carrier vehicle according to claim 23 wherein said fluorocarbon is selected from the group consisting of 1,1,1,3,3-pentafluorobutane, 1,1,1,3,3-pentachlorobutane, 1,1-dichloro-1,3-difluorobut-2-ene, 1-chloro-1,1,3-trifluorobut-2-ene, perfluorobutylethane, hexafluoroisobutylene, hexafluoroisopropano, perfluoromethane, perfluoroethane, perfluoropropane, perfluorobutane, perfluorocyclobutane, perfluoropentane, perfluorohexane, perfluorooctane, perfluorooctyl bromide, perfluorooctyl iodide, perfluorodecalin and 5 perfluoronapthalene.

27. The liquid carrier vehicle according to claim 26 wherein said fluorocarbon is selected from the group consisting of perfluorooctyl bromide and perfluorodecalin.

28. The liquid carrier vehicle according to claim 27 wherein said fluorocarbon is present in the carrier vehicle at from about 50% v/v to about 95% v/v.

29. The liquid carrier vehicle according to claim 28 wherein said fluorocarbon is present in said carrier vehicle at from about 65% v/v to about 95% v/v.

30. The liquid carrier vehicle according to claim 23 wherein said co-solvent is selected from the group consisting of alcohols, ethers and alkyl sulfoxides.

31. The liquid carrier vehicle according to claim 30 wherein said co-solvent is ethanol.

32. The liquid carrier vehicle according to claim 31 wherein said ethanol is present in said carrier vehicle at from about 5% v/v to about 50% v/v.

33. The liquid carrier vehicle according to claim 32 wherein said ethanol is present in the carrier vehicle at from about 5% v/v to about 35% v/v.

34. The liquid carrier vehicle according to claim 23 wherein said phospholipid is selected from the group consisting phosphatidic acid, phosphatidylethanolamine, lecithin, phosphatydylglycerol, diphosphatydylglycerol, dipalmitoylphosphatidylcholine, 1-palmitoyl-2-oleoyl-phosphatidylglycerol, dipalmitoylphosphatidylglycerol, as well as mixtures of such phospholipids.

35. The liquid carrier vehicle according to claim 34 wherein said phospholipid is selected from the group consisting of lecithin, dipalmitoylphosphatidylcholine and 1-palmitoyl-2-oleoyl-phosphatidylglycerol.

36. The liquid carrier vehicle according to claim 35 wherein said phospholipid is present in the carrier vehicle at from about 0.1% w/v to about 2.0% w/v.

37. The liquid carrier vehicle according to claim 36 wherein said phospholipid is present in the carrier vehicle at from about 0.1% w/v to about 1.0 w/v.

38. The liquid carrier vehicle according to claim 23 wherein said liquid carrier vehicle contains a pharmaceutically acceptable salt and wherein said salt is present in said carrier vehicle at from about 0.05% w/v to about 5.0% w/v.

39. The liquid carrier vehicle according to claim 38 wherein said salt is selected from the group consisting of benzalkonium chloride, sodium chloride and potassium chloride or mixtures thereof.

40. The liquid carrier vehicle according to claim 23 wherein said pharmaceutically acceptable excipient is selected from the group comprising polyols, antioxidants, anti-microbials, pH adjusting agents, and viscosity adjusting agents.

41. The liquid carrier vehicle according to claim 40 wherein said pharmaceutically acceptable excipient is selected from the group consisting essentially of propylene glycol, glycerol, polyvinyl alcohol, polyethylene glycol having an average molecular weight between about 200 and 4000, Vitamin E, Vitamin E TPGS, ascorbic acid, methyl paraben, ethyl paraben, sodium hydroxide, hydrochloric acid, polyvinyl alcohol and polyvinyl pyrrolidone.

42. The liquid carrier vehicle according to claim 41 wherein said pharmaceutically acceptable excipient is present in said carrier vehicle at from 0.05% w/v% to about 5.0% w/v.

43. The liquid carrier vehicle according to claim 42 wherein said pharmaceutically acceptable excipient is present in said carrier vehicle at from about 0.1% w/v% to about 2.5% w/v.

44. The liquid carrier vehicle according to claim 23 consisting essentially of:
i) from about 50% v/v to about 95 v/v of a liquid fluorocarbon;
ii) from about 5% v/v to about 50% v/v of a co-solvent;
iii) from about 0.1% w/v to about 2.0% w/v of a phospholipid; and
iv) from about 0.05% w/v to about 5.0% w/v of a pharmaceutically acceptable excipient.

45. The liquid carrier vehicle according to claim 44 consisting essentially of:
i) from about 65% v/v to about 95 v/v of a liquid fluorocarbon;
ii) from about 5.0% v/v to about 35% v/v of a co-solvent;
iii) from about 0.1% w/v to about 1.0% w/v of a phospholipid; and
iv) from about 0.1% w/v to about 2.5% w/v of a pharmaceutically acceptable excipient.

46. A method for delivering a pharmaceutically active agent to the respiratory tract of a patient in need of treatment comprising the steps of:
a) preparing a liquid carrier vehicle consisting essentially of:
i. from about 30% v/v to about 99 v/v of a liquid fluorocarbon;
ii. from about 1% w/v to about 70% w/v of a co-solvent;
iii. from about 0.0% w/v to about 10% w/v of a phospholipid;
iv. from about 0.0% w/v to about 10.0 w/v of a pharmaceutically acceptable excipient;
b) dissolving or suspending a pharmaceutically effective amount of said active agent in said liquid carrier vehicle;
c) producing an aerosol of said solution or suspension using an EHD spraying/aerosolization means; and
d) administering said aerosol to the pulmonary tract of said patient via inhalation of said aerosol;
wherein said liquid carrier vehicle has a surface tension of from about 15 dyne/cm to about 40 dyne/cm.

47. The method according to claim 46 wherein said fluorocarbon is present in said carrier vehicle at from about 50% v/v to about 95% v/v.

48. The method according to claim 47 wherein said fluorocarbon is present in said carrier vehicle at from about 65% v/v to about 95% v/v.

49. The method according to claim 46 wherein said fluorocarbon is selected from the group consisting 1,1,1,3,3-pentafluorobutane, 1,1,1,3,3-pentachlorobutane, 1,1-dichloro-1,3-difluorobut-2-ene, 1-chloro-1,1,3-trifluorobut-2-ene, perfluorobutylethane, hexafluoroisobutylene, hexafluoroisopropano, perfluoromethane, perfluoroethane, perfluoropropane, perfluorobutane, perfluorocyclobutane, perfluoropentane, perfluorohexane, perfluorooctane, perfluorooctyl bromide, perfluorooctyl iodide, perfluorodecalin and perfluoronapthalene.

50. The method according to claim 49 wherein said fluorocarbon is selected from the group consisting of perfluorooctyl bromide and perfluorodecalin.

51. The method according to claim 50 wherein said fluorocarbon is present in the carrier vehicle at from about 50% v/v to about 95% v/v.

52. The method according to claim 51 wherein said fluorocarbon is present in said carrier vehicle at from about 65% v/v to about 95% v/v.

53. The method vehicle according to claim 46 wherein said co-solvent is selected from the group consisting of alcohols, ethers and alkyl sulfoxides.

54. The method according to claim 53 wherein said co-solvent is ethanol.

55. The method according to claim 54 wherein said ethanol is present in said carrier vehicle at from about 5% v/v to about 50% v/v.

56. The method according to claim 55 wherein said ethanol is present in the carrier vehicle at from about 5% v/v to about 35% v/v.

57. The method according to claim 46 wherein said phospholipid is selected from the group consisting of phosphatidic acid, phosphatidylethanolamine, lecithin, phosphatydylglycerol, diphosphatydylglycerol, dipalmitoylphosphatidylcholine, 1-palmitoyl-2-oleoyl-phosphatidylglycerol, dipalmitoylphosphatidylglycerol, as well as mixtures of such phospholipids.

58. The method according to claim 57 wherein said phospholipid is selected from the group consisting lecithin, dipalmitoylphosphatidylcholine and 1-palmitoyl-2-oleoyl-phosphatidylglycerol.

59. The method according to claim 57 wherein said phospholipid is present in the carrier vehicle at from about 0.1% w/v to about 2.0% w/v.

60. The method according to claim 59 wherein said phospholipid is present in the carrier vehicle at from about 0.1% w/v to about 1.0 w/v.

61. The method according to claim 46 wherein said carrier vehicle contains a pharmaceutically acceptable salt and wherein said salt is present in said carrier vehicle at from about 0.05% w/v to about 5.0% w/v.

62. The method according to claim 61 wherein said salt is selected from the group consisting of benzalkonium chloride, sodium chloride and potassium chloride or mixtures thereof.

63. The method according to claim 46 wherein said pharmaceutically acceptable excipient is selected from the group comprising polyols, antioxidants, anti-microbials, pH adjusting agents, and viscosity adjusting agents.

64. The method according to claim 63 wherein said pharmaceutically acceptable excipient is selected from the group consisting essentially of propylene glycol, glycerol, polyvinyl alcohol, polyethylene glycol having an average molecular weight between about 200 and 4000, Vitamin E, Vitamin E TPGS, ascorbic acid, methyl paraben, ethyl paraben, sodium hydroxide, hydrochloric acid, polyvinyl alcohol and polyvinyl pyrrolidone.

65. The method according to claim 63 wherein said pharmaceutically acceptable excipient is present in said carrier vehicle at from 0.05% w/v% to about 5.0% w/v.

66. The method according to claim 65 wherein said pharmaceutically acceptable excipient is present in said carrier vehicle at from about 0.1% w/v% to about 2.5% w/v.

67. The method according to claim 46 consisting essentially of:
i) from about 50% v/v to about 95 v/v of a liquid fluorocarbon;
ii) from about 5% v/v to about 50% v/v of a co-solvent;
iii) from about 0.1% w/v to about 2.0% w/v of a phospholipid;
iv) from about 0.05% w/v to about 5.0% w/v of a pharmaceutically acceptable excipient.

68. The method according to claim 46 consisting essentially of:
i) from about 65% v/v to about 95 v/v of a liquid fluorocarbon;
ii) from about 5.0% v/v to about 35% v/v of a co-solvent;
iii) from about 0.1% w/v to about 1.0% w/v of a phospholipid;
iv) from about 0.1% w/v to about 2.5% w/v of a pharmaceutically acceptable excipient.
